(19) United States
(12) Patent Application Publication
(10) Pub. No.: US 2022/0319644 A1
(43) Pub. Date: Oct. 6, 2022

(54) SYSTEMS AND METHODS FOR DETECTING FRAUDULENT PRIOR AUTHORIZATION REQUESTS

(71) Applicant: Change Healthcare Holdings, LLC, Nashville, TN (US)

(72) Inventors: Allen Perry Saunders, Philadelphia, PA (US); Thomas House, Hanson, MA (US); Sita Singh, Burlington, MA (US); Sergei Bobronnikov, Wayland, MA (US); Prakash Varigonda, Sharon, MA (US); Leonard Kierstead, Upton, MA (US); Lap Yee, Medford, MA (US)

(21) Appl. No.: 17/216,788

(22) Filed: Mar. 30, 2021

(51) Int. Cl. G16H 10/60; G06Q 40/08; G06N 5/00; G16H 20/00; G06Q 50/26

(52) U.S. Cl. CPC G16H 10/60; G06Q 40/08; G06N 5/003; G16H 20/00; G06Q 50/265

(57) ABSTRACT

A system for scoring automatic prior authorization requests is provided. The system may receive a request for automatic prior authorization from a medical provider, and in response may provide the medical provider with a plurality of questions. Once answers to the questions are received from the medical provider, the system may compute a score for the request that relates to the overall trustworthiness of the request. If the score satisfies a threshold, the request is flagged for further review. Otherwise, the request may be processed as a normal request. The score may be based on a variety of heuristics that indicate that a request may be fraudulent or constructed to gain an automatic prior authorization. The heuristics may consider information such as the speed at which the questions were answered, the particular answers given to one or more of the questions, and the request history of the medical provider.

### BACKGROUND

Prior authorization, also known as pre-authorization, is a requirement from payors (e.g., health insurance companies) for medical providers to obtain approval before the payors will cover the costs of a specific medicine, medical device, or procedure for a patient. Generally, a medical provider (e.g., doctor) will provide a request for prior authorization to a payor entity before performing a surgery. As part of the request, the payor entity may ask the medical provider a series of questions about the patient and their medical conditions. The payor entity may then either approve the request, and provide prior authorization, or may deny the request.

As may be appreciated, the process to receive prior authorization from a payor may be a time consuming process because it uses a human reviewer. One solution is the use of automated prior authorization. In automated prior authorization, when a request is received, a computer may ask the medical provider a series of questions. Depending on the answers, the computer may automatically approve the request or may forward the request to a human reviewer for further analysis.

While automatic prior authorization is fast, it is associated with its own problems. In particular, automatic prior authorization may be susceptible to gaming or even fraud by medical providers. For example, a medical provider who performs a particular type of surgery may learn the answers that lead to an automatic prior authorization and may “fudge” patient data to achieve an automatic prior authorization. Such fraud may be expensive to a payor and may be difficult to remedy after the prior authorization has been provided and the medical items has been performed by the medical provider or received by the patient.

### SUMMARY

In an embodiment, a system for scoring automatic prior authorization requests is provided. The system may receive a request for automatic prior authorization from a medical provider, and in response may provide the medical provider with a plurality of questions. The questions may be the questions used by a payor to determine whether the automatic prior authorization should be granted. Once answers to the questions are received from the medical provider, the system may compute a score for the request that relates to the overall trustworthiness of the request. If the score satisfies a threshold, the request is flagged for further review. Otherwise, the request may be processed as a normal request. The score may be based on a variety of heuristics that indicate that a request may be fraudulent or constructed to gain an automatic prior authorization. The heuristics may consider information such as the speed at which the questions were answered, the particular answers given to one or more of the questions, and the request history of the medical provider. Alternatively, the score may be generated using a model that is trained to identify fraudulent requests.

In an embodiment, a method for scoring prior authorization requests is provided. The method includes: receiving a request for prior authorization for a medical item from a medical provider by a computing device through a network; in response to the request, providing one or more questions associated with the medical item to the medical provider by the computing device through the network; receiving answers to the one or more questions from the medical provider through the network; based on the received answers, information associated with the medical provider, and information associated with the request, assigning a score to the request by the computing device; determining whether the score satisfies a threshold by the computing device; and if it is determined that the score satisfies a threshold, determining that the request is fraudulent and providing the request and answers to a payor entity through the network.

Embodiment may include some or all of the following features. The payor entity may be an insurance provider. The threshold may be provided by the payor entity. Assigning the score to the request may include using a model to generate the score using some or all of the received answers, the information associated with the medical provider, and the information associated with the request. Assigning the score to the request may include applying one or more heuristics to some or all of the received answers, the information associated with the medical provider, and the information associated with the request. The heuristics may include one or more of a rush heuristic, a similarity heuristic, a backtracking heuristic, an approval rate heuristic, an abandonment heuristic, and an outlier answer heuristic. If it is determined that the score does not satisfy the threshold, processing the request for prior authorization based on the received answers. The medical item may include one or more of a medicine, a medical test, or a medical procedure.

In an embodiment, a method for detecting fraudulent prior authorization requests is provided. The method includes: receiving a request for prior authorization for a medical item from a medical provider by a computing device through a network; in response to the request, providing one or more questions associated with the medical item to the medical provider by the computing device through the network; receiving answers to the one or more questions from the medical provider through the network; based on the received answers, information associated with the medical provider, and information associated with the request, determining whether the request is a fraudulent request by the computing device; and if it is determined that the request is a fraudulent request, providing the request and answers to a payor entity through the network.

Embodiments may include some or all of the following features. The payor entity may be an insurance provider. Determining that the request is a fraudulent request may include: based on the received answers, the information associated with the medical provider, and the information associated with the request, assigning a score to the request; determining whether the score satisfies a threshold; and if it is determined that the score satisfies a threshold, determining that the request is a fraudulent request. Assigning the score to the request may include using a model to generate the score using some or all of the received answers, the information associated with the medical provider, and the information associated with the request. Assigning the score to the request may include applying one or more heuristics to some or all of the received answers, the information associated with the medical provider, and the information associated with the request. The heuristics may include one or more of a rush heuristic, a similarity heuristic, a backtracking heuristic, an approval rate heuristic, an abandonment heuristic, and an outlier answer heuristic. If it is determined that the request is not fraudulent, processing the request for prior authorization based on the received answers. The medical item may include one or more of a medicine, a medical test, or a medical procedure.

This summary is provided to introduce a selection of concepts in a simplified form that are further described below in the detailed description. This summary is not intended to identify key features or essential features of the claimed subject matter, nor is it intended to be used to limit the scope of the claimed subject matter.

### BRIEF DESCRIPTION OF THE DRAWINGS

The foregoing summary, as well as the following detailed description of illustrative embodiments, is better understood when read in conjunction with the appended drawings. For the purpose of illustrating the embodiments, there is shown in the drawings example constructions of the embodiments; however, the embodiments are not limited to the specific methods and instrumentalities disclosed. In the drawings:

FIG. 1 is an illustration of an exemplary environment for processing automatic prior authorization requests;

FIG. 2 is an illustration of a method for scoring a request for prior authorization;

FIG. 3 is an illustration of a method for scoring a request for prior authorization;

and

FIG. 4 shows an exemplary computing environment in which example embodiments and aspects may be implemented.

### DETAILED DESCRIPTION

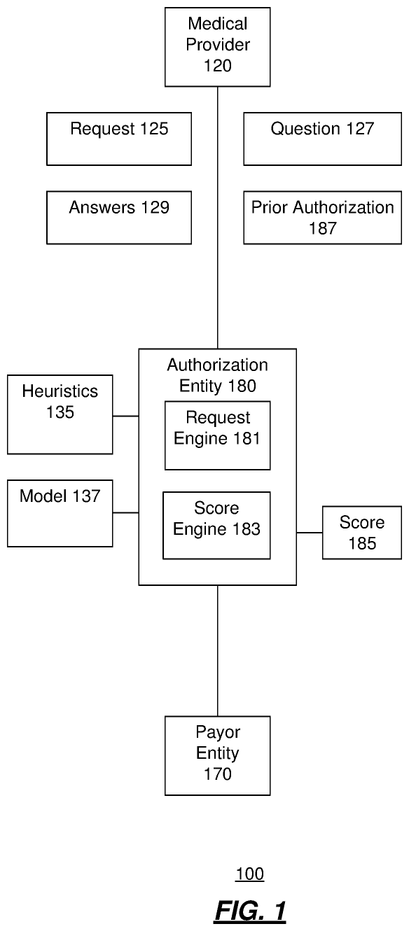

FIG. 1 is an illustration of an exemplary environment 100 for processing automatic prior authorization requests. A prior authorization request 125 (also referred to as pre-authorization requests) is a request that a payor entity 170 agree to pay for a medical item in the future. Medical item as used herein may include medication (e.g., prescription drugs) and medical procedures (e.g., medical tests and surgical procedures). The medical item may be provided by a medical providers 120 such as a doctor, hospital, or clinic. Other types of medical or healthcare providers may be supported. Payor entities 170 may include insurance companies, government entities, and third-party payers. Other types of payers may be supported.

In order to speed the approval of requests 125, the environment 100 may include an authorization entity 180. The authorization entity 180 may include a request engine 181 that receives a request 125 for prior authorization from a medical provider 120, and in response may provide one or more questions 127 that have been provided by the payor entity 170 for the purpose of automatically approving the request 125. The questions 127 may be specific to the medical item that is the subject of the request 125. The questions 127 may include questions about the patient and the medical history of the patient.

For example, a medical provider 120 may send a prior authorization request 125 to an authorization entity 180 for a hip replacement through a network (e.g., the internet). In response, the request engine 181 of the authorization entity 180 may send questions 127 to the medical provider 120 that were specified by the payor entity 170 for requests 125 related to hip replacements. The questions 127 may request information such as the age of the patient, the height and weight of the patient, other treatments, or procedures that the patient had related to the hip injury, measurements or data taken from one or more tests or diagnostic images performed on the patient, and any medications taken by the patient. Other types of questions 127 may be used.

The medical provider 120 may provide answers 129 to the questions 127, and the request engine 181 may process the answers 129 to determine if the prior authorization 187 may be automatically granted or denied. Depending on the embodiment, the payor entity 170 may have provided guidelines or rules that may be used to process the answers 129. If the request engine 181 approves the request 125, the request engine 181 may provide the prior authorization 187 to the medical provider 120 through the network. If the request engine 181 does not approve, the request 125 may be denied and/or provided to the payor entity for a manual review.

As may be appreciated, while automatically approving prior authorizations 187 is more convenient than manually approving prior authorizations, they are also more susceptible to fraud. In particular, medical providers 120 may learn, through trial and error or experience, what answers 129 are likely to result in an automatic prior authorization 187 for a particular medical item and may change or adjust the corresponding answers 129 for a patient to fraudulently receive an automatic prior authorization 187. For example, a medical provider 120 may learn that a patient having a particular weight or symptom is more likely to be approved for a surgical procedure. The medical provider 120 may then adjust the weight of the patient or lie about the presence of the symptom in their answers 129.

As another example, a medical provider 120 may be rejected for an automatic prior authorization 187. In response, the provider 120 may resubmit the request 125 after adjusting one or more of the answers 129 used in the previous request 125. The medical provider 120 may continue to resubmit the request 125 with adjusted answers 129 until an automatic prior authorization 187 is granted.

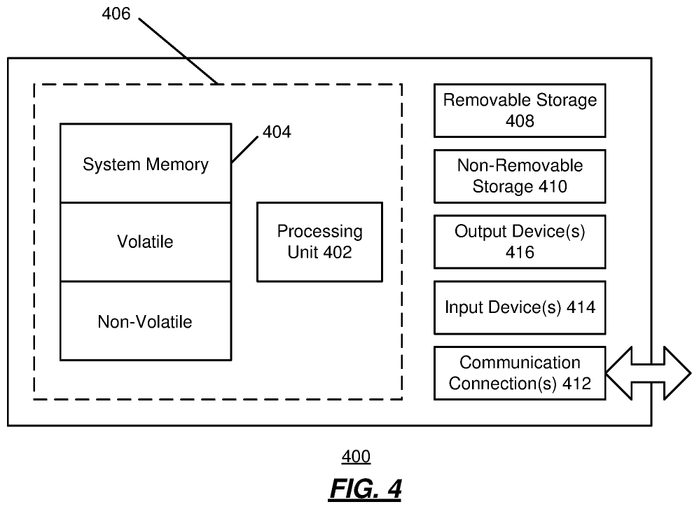

To prevent fraudulent requests, the authorization entity 180 may further include a score engine 183. The request engine 181 and the score engine 183 may be implemented together or separately using one or more general purpose computing devices such as the computing device 400 illustrated with respect to FIG. 4.

The score engine 183 may determine whether or not a request 125 for prior authorization is likely a fraudulent request, and in response to the determination the score engine 183 may deny the request 125 and may provide the fraudulent request 125 to the payor entity 170 for further investigation. Previously, fraudulent requests 125 for prior authorization 187 were detected through auditing of selected requests 125. However, these fraudulent requests 125 were not detected until after the prior authorizations 187 were approved and the associated medical procedure was completed. In contrast, the proposed method for detecting fraudulent requests 125 is preformed before the prior authorization 187 is granted and the medical procedure is performed. Thus, the proposed methods save both time and money when compared to prior art methods.

The score engine 183 may generate a score 185 for a request 125 based on a variety of information about the request 125. The information may include the particular answers 129 given for each question 127, the amount of time that the medical provider 120 spent answering each question 127, the frequency or number of requests 125 that have been recently submitted by the medical provider 120, and any other information about the medical provider 120 that may be relevant for determining whether a request 125 is fraudulent.

The score engine 183 may compare the score 185 generated for a request 125 to a threshold, and if the threshold is satisfied (e.g., the score 185 is above the threshold), the score engine 183 may determine that the request 125 is a fraudulent request. Depending on the embodiment, the threshold may be provided by the payor entity 170.

In some embodiments, the score engine 183 may assign the score 185 to a request 181 using one or more heuristics 135. The heuristics 135 may be rules or guidelines for identifying fraudulent requests 125 developed by observing those requests 125 that were later determined to be fraudulent. Example heuristics 135 that may be used by the score engine 183 include a rush heuristic, a similarity heuristic, a backtracking heuristic, an approval rate heuristic, an abandonment heuristic, and an outlier answer heuristic.

The rush heuristic is based on the amount of time that the medical provider 120 spent answering the questions 127 of the request 125. Generally, if a medical provider 120 answers the questions 127 for a request 125 too quickly it may indicate that the provider 120 is not taking the time to answer each question accurately or truthfully by looking in the file associated with the patient, but is instead quickly answering some of the questions 127 using pre-determined answers 129 that are known to lead to approvals. The amount of time that is considered to trigger the rush heuristic may be determined by observing the average amount of time spent by medical providers 120 answering particular questions 127.

The similarity heuristic is based on the repetition of a particular answer 129 across multiple requests 125 for a medical provider 120. If there is a large amount of repetition of a particular answer 129 to a question it may indicate that the provider 120 is not providing truthful answers 129 but is instead providing answers 129 that they believe will lead to an automatic prior authorization 187. For example, a provider 120 may provide the same weight for multiple requests 125. The number of repetitive answers 129 needed to trigger the similarity heuristic may be determined by observing the typical answer duplication across fraudulent and non-fraudulent requests 125.

The backtracking heuristics is based on the observation that when a provider 120 makes multiple changes to answers 129 when completing a request 125 it may indicate that the provider 120 is fraudulently changing answers 129 to find a combination of answers 129 that may result in a prior authorization 187. The number of changes needed to trigger the backtracking heuristic may be determined by observing the typical number of changes made across fraudulent and non-fraudulent requests 125.

The approval rate heuristic is based on the observation that most medical providers 120 do not receive approval for all of their requests 125 for prior authorization. Therefore, if a medical provider 120 is receiving a very high approval rate for requests 125 it may indicate that some of the requests 125 are fraudulent. For example, an average approval rate for medical providers 120 may be 90%. If a medical provider 120 has an approval rate of 95% it may indicate that some of the requests 125 are fraudulent. Depending on the embodiment, the approval rate applied may be for requests 125 for all medical items, or specific to requests 125 for particular medial items (e.g., prescription requests vs. surgical procedure requests). The approval percentage needed to trigger the approval rate heuristic may be determined by observing the typical approval rate for medical providers 120 over time.

The abandonment heuristic is based on the observation that when a medical provider 120 abandons a request 125, but then resubmits the abandoned request 125 at a later time, it may indicate that the request 125 is a fraudulent request. For example, the provider 120 may abandon the request 125 while they determine the best combination of answers 129 that will result in an approval. What constitutes an abandoned request for purposes of the score 185 may be determined by observing previously abandoned requests 125 that turn into fraudulent requests 125. The outlier heuristic is based on the observation that certain answers 129 may be very infrequently received from medical providers 120 in response to certain questions 127. Accordingly, if such an answer 129 is received from a medical provider 120 it may indicate that the request 125 is a fraudulent request 125. The outlier answers needed to trigger the outlier heuristic may be determined by observing the typical answers 129 given in non-fraudulent requests 125.

In one embodiment, the authorization entity 180 may provide a portal webpage that the medical provider 120 may access when submitting a request 125. The request engine 181 may present the questions 127 through the portal, and the provider 120 may use the portal to answer the questions 127. The questions 127 presented in the portal may change dynamically based on the answers 129 submitted by the provider 120. The score engine 183 may use the portal to record information used to determine the score 185 including the speed at which the provider answered each question 127, the answers 129 provided, and the number of answers 129 that were changed by the medical provider 120.

The score engine 183 may determine a score 183 for a request 125 by combining each of the heuristics 135. For example, each of the rush, backtrack, similarity, outlier, and approval rate heuristics may generate a score 185, and the score engine 183 may combine the scores 185 to generate an overall score 185 for the request 125. The scores 185 may be combined using a weighted sum where each heuristic 135 is assigned a different weight by the score engine 183 and/or the payor entity 170.

Other information may be considered by the score engine 183 when generating a score 183. Example information may include the history of the medical provider 120 (i.e., has the medical provider 120 submitted fraudulent requests before), and the particular medical item associated with the request 125 (i.e., is the medical item one that is commonly associated with fraudulent requests 125).

In some implementations, the score engine 183 may further consider the medical history of the patient when generating the score 183. In particular, the score engine 183 may attempt to match or verify some of the information in the answer 129 and/or the request 125 with the medical history of the patient. If too much information does not match the medical history or is absent from the medical history, then the request 125 is likely fraudulent and the score 183 may be increased.

For example, if the weight, age, height, or other information associated with an answer 129 does not match the medical history of the patient, then the request 125 may be fraudulent. As another example, if the answer 129 indicates that the patient has had six weeks of physical therapy, but the medical history of the patient does not include any claims or other evidence of physical therapy, then the request 125 may be fraudulent.

In an alternative embodiment, the score engine 183 may generate a score 185 for a request 125 using a model 137. The model 137 may take as an input various information about a request 125 such as the answers 129 provided for the questions 127, how long each question 127 took to complete, and whether there was any backtracking. Other information such as the medical item associated with the request and the medical provider 120 associated with the request may also be provided. The model 137 may then use some or all of the provided information to determine a score 185 for the request 125 that represents the probability that the request 125 is fraudulent.

The model 137 may be generated using machine learning. For example, the model 137 may be trained using a set of previously received requests 125 (and associated information) that are known to be legitimate, and a set of previously received requests 125 (and associated information) that are known to be fraudulent. Any method for training a model 137 may be used.

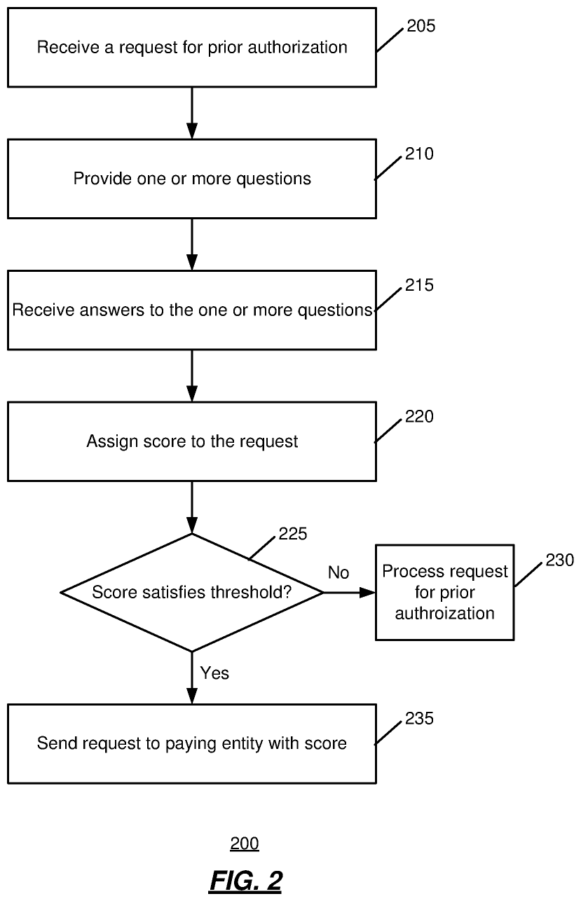

FIG. 2 is an illustration of a method 200 for scoring a request for prior authorization. The method 200 may be performed by the authorization entity 180.

At 205, a request for prior authorization is received. The request 125 may be received by the request engine 181 of the authorization entity 180. The request 125 may be associated with a medical item such as a medical procedure or a medication. The request for prior authorization 125 may be a request for an automatic prior authorization (i.e., a request that is approved automatically by a computing device without review by a human approver). Depending on the embodiment, the request 125 may be received by the authorization entity 180 through a portal or webpage provided or hosted by the authorization entity 180.

At 210, one or more questions are provided. The one or more questions 127 may be provided by the request engine 181 to the medical provider 120 in the portal or webpage that was used to provide the request. The questions 127 may be based on the medical item that was referenced in the request 125 for prior authorization.

At 215, answers to the provided questions are received. The answers 129 may be received through the portal or webpage from the medical provider 120. In some embodiments, the answers 129 may be received by the request engine 181 after all of the answers 129 have been completed by the medical provider 120. Alternatively, the answers 129 may be received as they are entered by the medical provider 120. In such embodiments, additional questions 127 may be provided to the medical provider 120 depending on the answers 129 that are received.

At 220, a score is assigned to the request. The score 185 may be assigned to the request 125 for prior authorization by the score engine 183. The score 185 may be based on information about the request 125 (e.g., the answers 129, the medical item, the amount of time the medical provider took to complete each request, answers 129 that were changed by the medical provider 120, the medical provider 120, and the request 125 history of the medical provider). In some embodiments, the score engine 183 may assign the score 185 using the information associated with the request 125 and one or more heuristics 135 including, but not limited to, the rush heuristic, the similarity heuristic, the backtracking heuristic, the approval rate heuristic, and the outlier answer heuristic. Other heuristics may be used.

At 225, a determination is made as to whether the score satisfies a threshold. The determination may be made by the score engine 183 using a threshold that was provided by the payor entity 170 (i.e., the insurance provider or third-party provider that is providing the prior authorization 187 and will pay for the medical item). The threshold may be satisfied when the assigned score 185 is above the threshold. If the threshold is not satisfied, the method 200 may continue at 230. If the threshold is satisfied, the method 200 may continue at 235.

At 230, the request for prior authorization is processed. The request 125 may be processed by the request engine 181. Because the score 185 did not satisfy the threshold, the request 125 may be presumed to be true and not fraudulent. The request engine 181 may process the request 125, including answers 129, according to rules or guidelines provided by the payor entity 170. Depending on the outcome of the processing, the request engine 181 may approve the request 125, may deny the request 125, or may send the request 125 to the payor entity 170 for a manual in-person review. If the request engine 181 approves the request 125 a prior authorization 187 may be provided to the medical provider 120.

At 235, the request is sent to the payor entity. The request 125 may be sent to the payor entity 170 along with the generated score 185, the answers 129, and other information. Because the score 185 satisfied the threshold it was presumed to be a fraudulent request 125. The payor entity 170 may then review the request 125 to determine if it was in fact fraudulent. If the request 125 is determined to be not fraudulent, the payor entity 170 may return to the request 125 to the authorization entity 180 for processing.

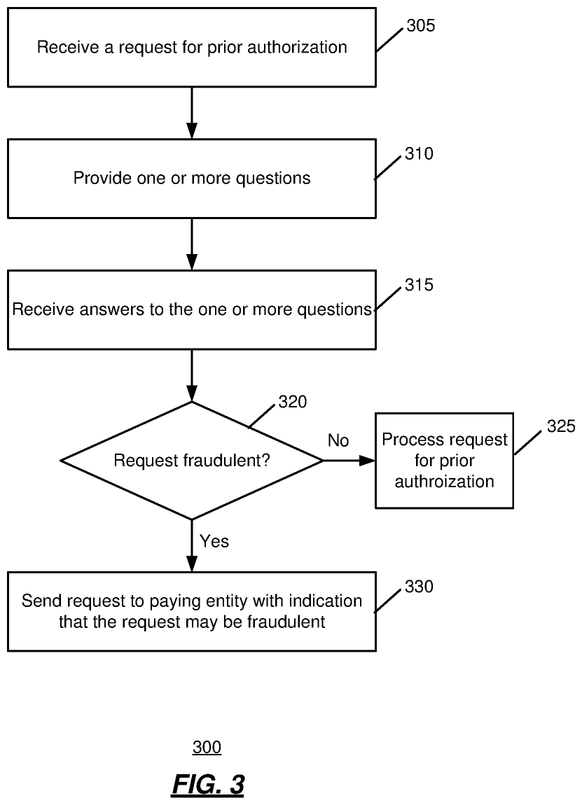

FIG. 3 is an illustration of a method 300 for scoring a request for prior authorization. The method 300 may be performed by the authorization entity 180.

At 305, a request for prior authorization is received. The request 125 may be received by the request engine 181 of the authorization entity 180. The request 125 may be associated with a medical item such as a medical procedure or a medication. Depending on the embodiment, the request 125 may be received by the authorization entity 180 through a portal or webpage provided or hosted by the authorization entity 180.

At 310, one or more questions are provided. The one or more questions 127 may be provided by the request engine 181 to the medical provider 120 in the portal or webpage that was used to provide the request. The questions 127 may be based on the medical item that was referenced in the request 125 for prior authorization.

At 315, answers to the provided questions are received. The answers 129 may be received through the portal or webpage from the medical provider 120.

At 320, a determination is made as to whether the request is a fraudulent request. The determination may be made by the score engine 183 using the received answers 129, information associated with the medical provider 120, and information associated with the request 125. In some embodiments, the score engine 183 may use a model 137 to generate a score 185 that represents a probability that the request 125 is fraudulent. If the score is above a threshold, then the score engine 183 may determine that the request 125 is likely fraudulent. If the request 125 is likely fraudulent, the method 300 may continue at 330. If the request 125 is not fraudulent, the method 300 may continue at 325.

At 325, the request for prior authorization is processed. The request 125 may be processed by the request engine 181. Because the request 125 was determined to be not fraudulent, the request engine 181 may process the request 125, including answers 129, according to rules or guidelines provided by the payor entity 170. Depending on the outcome of the processing, the request engine 181 may approve the request 125, may deny the request 125, or may send the request 125 to the payor entity 170 for a manual in-person review. If the request engine 181 approves the request 125 a prior authorization 187 may be provided to the medical provider 120.

At 330, the request is sent to the payor entity. The request 125 may be sent to the payor entity 170 along with the generated score 185, the answers 129, and other information. Because the score 185 satisfied the threshold, it was presumed to be a fraudulent request 125. The payor entity 170 may then review the request 125 to determine if it was in fact fraudulent. If the request 125 is determined to be not fraudulent, the payor entity 170 may return to the request 125 to the authorization entity 180 for processing.

FIG. 4 shows an exemplary computing environment in which example embodiments and aspects may be implemented. The computing device environment is only one example of a suitable computing environment and is not intended to suggest any limitation as to the scope of use or functionality.

Numerous other general purpose or special purpose computing devices environments or configurations may be used. Examples of well-known computing devices, environments, and/or configurations that may be suitable for use include, but are not limited to, personal computers, server computers, handheld or laptop devices, multiprocessor systems, microprocessor-based systems, network personal computers (PCs), minicomputers, mainframe computers, embedded systems, distributed computing environments that include any of the above systems or devices, and the like.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

With reference to FIG. 4, an exemplary system for implementing aspects described herein includes a computing device, such as computing device 400. In its most basic configuration, computing device 400 typically includes at least one processing unit 402 and memory 404. Depending on the exact configuration and type of computing device, memory 404 may be volatile (such as random access memory (RAM)), non-volatile (such as read-only memory (ROM), flash memory, etc.), or some combination of the two. This most basic configuration is illustrated in FIG. 4 by dashed line 406.

Computing device 400 may have additional features/functionality. For example, computing device 400 may include additional storage (removable and/or non-removable) including, but not limited to, magnetic or optical disks or tape. Such additional storage is illustrated in FIG. 4 by removable storage 408 and non-removable storage 410.

Computing device 400 typically includes a variety of computer readable media. Computer readable media can be any available media that can be accessed by the device 400 and includes both volatile and non-volatile media, removable and non-removable media.

Computer storage media include volatile and non-volatile, and removable and non-removable media implemented in any method or technology for storage of information such as computer readable instructions, data structures, program modules or other data. Memory 404, removable storage 408, and non-removable storage 410 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by computing device 600. Any such computer storage media may be part of computing device 400.

Computing device 400 may contain communication connection(s) 412 that allow the device to communicate with other devices. Computing device 400 may also have input device(s) 414 such as a keyboard, mouse, pen, voice input device, touch input device, etc. Output device(s) 416 such as a display, speakers, printer, etc. may also be included. All these devices are well known in the art and need not be discussed at length here.

It should be understood that the various techniques described herein may be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

Although exemplary implementations may refer to utilizing aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be effected across a plurality of devices. Such devices might include personal computers, network servers, and handheld devices, for example.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A method for scoring prior authorization requests comprising:
receiving a request for prior authorization for a medical item from a medical provider by a computing device through a network;
in response to the request, providing one or more questions associated with the medical item to the medical provider by the computing device through the network;
receiving answers to the one or more questions from the medical provider through the network;
based on the received answers, information associated with the medical provider, and information associated with the request, assigning a score to the request by the computing device;
determining whether the score satisfies a threshold by the computing device; and
if it is determined that the score satisfies a threshold, determining that the request is fraudulent and providing the request and answers to a payor entity through the network.

2. The method of claim 1, wherein the payor entity is an insurance provider.

3. The method of claim 1, wherein the threshold is provided by the payor entity.

4. The method of claim 1, wherein assigning the score to the request comprises using a model to generate the score using some or all of the received answers, the information associated with the medical provider, and the information associated with the request.

5. The method of claim 1, wherein assigning the score to the request comprises applying one or more heuristics to some or all of the received answers, the information associated with the medical provider, and the information associated with the request.

6. The method of claim 1, wherein the heuristics comprise one or more of a rush heuristic, a similarity heuristic, a backtracking heuristic, an approval rate heuristic, an abandonment heuristic, and an outlier answer heuristic.

7. The method of claim 1, wherein if it is determined that the score does not satisfy the threshold, processing the request for prior authorization based on the received answers.

8. The method of claim 1, wherein the medical item comprises one or more of a medicine, a medical test, or a medical procedure.

9. A method for detecting fraudulent prior authorization requests comprising:
receiving a request for prior authorization for a medical item from a medical provider by a computing device through a network;
in response to the request, providing one or more questions associated with the medical item to the medical provider by the computing device through the network;
receiving answers to the one or more questions from the medical provider through the network;
based on the received answers, information associated with the medical provider, and information associated with the request, determining whether the request is a fraudulent request by the computing device; and
if it is determined that the request is a fraudulent request, providing the request and answers to a payor entity through the network.

10. The method of claim 9, wherein the payor entity is an insurance provider.

11. The method of claim 9, wherein determining that the request is a fraudulent request comprises:
based on the received answers, the information associated with the medical provider, and the information associated with the request, assigning a score to the request;
determining whether the score satisfies a threshold; and
if it is determined that the score satisfies a threshold, determining that the request is a fraudulent request.

12. The method of claim 11, wherein assigning the score to the request comprises using a model to generate the score using some or all of the received answers, the information associated with the medical provider, and the information associated with the request.

13. The method of claim 11, wherein assigning the score to the request comprises applying one or more heuristics to some or all of the received answers, the information associated with the medical provider, and the information associated with the request.

14. The method of claim 13, wherein the heuristics comprise one or more of a rush heuristic, a similarity heuristic, a backtracking heuristic, an approval rate heuristic, an abandonment heuristic, and an outlier answer heuristic.

15. The method of claim 9, wherein if it is determined that the request is not fraudulent, processing the request for prior authorization based on the received answers.

16. The method of claim 9, wherein the medical item comprises one or more of a medicine, a medical test, or a medical procedure.

17. A system for scoring prior authorization requests comprising:
one or more processors;
a memory communicably coupled to the one or more processors and storing instructions that when executed by the one or more processors cause the one or more processors to:
receive a request for prior authorization for a medical item from a medical provider;
in response to the request, provide one or more questions associated with the medical item to the medical provider;
receive answers to the one or more questions from the medical provider;
based on the received answers, information associated with the medical provider, and information associated with the request, assign a score to the request;
determine whether the score satisfies a threshold; and
if it is determined that the score satisfies a threshold, determine that the request is fraudulent and provide the request and answers to a payor entity.

18. The system of claim 17, wherein assigning the score to the request comprises using a model to generate the score using some or all of the received answers, the information associated with the medical provider, and the information associated with the request.

19. The system of claim 18, wherein assigning the score to the request comprises applying one or more heuristics to some or all of the received answers, the information associated with the medical provider, and the information associated with the request.

20. The system of claim 18, wherein the heuristics comprise one or more of a rush heuristic, a similarity heuristic, a backtracking heuristic, an approval rate heuristic, an abandonment heuristic, and an outlier answer heuristic.
